(19) United States
(12) Patent Application Publication
(10) Pub. No.: US 2010/0145108 A1
(43) Pub. Date: Jun. 10, 2010

(54) Method for Purifying Product Mixtures From Transesterification Reactions

(75) Inventors: Michael Traving, Burscheid (DE); Johannes-Peter Schaefer, Kurten (DE); Rafael Warsitz, Essen (DE); Waldemar Widuch, Leverkusen (DE); Werner Bäcker, Wipperfurth (DE); Uwe Spörer, Oberhausen (DE)

Correspondence Address: Briscoe, Kurt G.;Norris McLaughlin & Marcus, PA, 875 Third Avenue, 8th Floor, New York, NY, 10022, US

(73) Assignee: BAYER TECHNOLOGY SERVICES GMBH, Leverkusen (DE)

(21) Appl. No.: 12/596,779

(22) PCT Filed: Apr. 10, 2008
(86) PCT No.: PCT/EP08/02828

§ 371 Date: Dec. 9, 2009

(30) Foreign Application Priority Data
Apr. 23, 2007 (DE) 10 2007 019 379.5

(51) Int. Cl. C07C 29/76

(52) U.S. Cl. 568/868

(57) ABSTRACT

The invention relates to an industrial process for purifying the product mixture of a transesterification reaction with a polar, electrolyte-containing organic phase, in which the electrolytes are removed from the permeating polar organic phase by means of a nanofiltration step.

The present invention relates to a process for purifying product mixtures from transesterification reactions comprising a polar, electrolyte-containing organic phase by a nanofiltration.

A variety of processes for purifying product mixtures, especially electrolyte-containing organic phases, has been known for some time. These processes are usually based on different thermal processes.

In general, in the case of such product mixtures, a product purification is carried out to remove the electrolytes in the form of a distillation. This procedure can also be employed on the industrial scale, provided that a sufficiently large column is used to prevent contamination of the organic phase by entrainment of a salt fraction.

A disadvantage of the distillation processes used to date is in particular the high energy expenditure specifically with regard to industrial scale use. In addition, the salt encrustations which occur in the distillation reservoir and also in the columns additionally cause the need to periodically interrupt the distilling operation and undertake cleaning of the equipment. In the industrial sector too, this is highly disadvantageous in the case of integration of the distilling operation into continuous preparation processes.

Another approach to the purification of salt-containing organic phases consists in the use of electrodialysis processes.

One example of the aforementioned principle based on the purification of glycerol from the preparation of fatty acid methyl esters is described in the publications of F. Schaffner and R. Audinos, “Electrodialysis of crude glycerol recovered after esterification of colza oil”, Proc. 7th World Filtration Congress, Budapest, 1998, pp. 629-633 and KR 2003 026 269 A. In this case, the salt-laden organic phase is conducted against salt-free water in an electrodialysis module. As a result of flow transfer, the salt ions are transported through the separating membrane and hence depleted in the organic phase.

A disadvantage of this method for working up electrolyte-containing organic product mixtures is, however, the high use of additional water, which is laden with electrolyte after the purification by dialysis. Specifically with regard to the ever greater strictness of environmental regulations, the disposal of the large amounts of salt-containing water in industrial processes is an important and costly aspect. A thermal workup of the additional water, as in the alternative method of distillation, causes further energy costs.

A further workup method consists in the desalinification of the organic phase by means of specific ion exchangers which exhibit good removal of the electrolytes at high throughput. The modern ion exchangers include resins and/or zeolites tailored for this purpose. The disadvantage, especially in the case of purification of glycerol from industrial biodiesel processes, is, however, the high salt burden of the glycerol from the process. As a result of this high content, a large volume of ion exchange resin is required, which also has to be regenerated again. The regeneration generates an amount of acidic water which has to be sent to a separate workup. This gives rise to further capital and operating costs for the assistants needed.

A further method described for the purification of electrolyte-containing product mixtures from transesterification reactions is the precipitation of the salts after removal of methanol and water by the addition of short-chain alcohols in a defined phase ratio of 1:0.3 to 1:4 (for example glycerol to alcohol). After intensive mixing, the salt can correspondingly be filtered out.

This procedure entails various disadvantages. Dewatering and removal of the methanol prior to the removal of the electrolyte is very energy-intensive, as already detailed above. The corresponding distillation apparatus additionally has a certain level of salt precipitation, which leads to the necessity of shutdowns in the process. Moreover, this procedure introduces a considerable additional volume into the process in the form of alcohol. This means an increased level of material and capital costs.

In recent times, specifically the production of biodiesel on the industrial scale has become increasingly economically significant. In this preparation, preferably methyl esters of the fatty acids present in purified or reprocessed oils are obtained therefrom by way of a transesterification reaction, and find use as fuel. Additionally obtained is glycerol, which is likewise used as a raw material, for example in the cosmetics industry. The transesterification reaction can either be acid- or base-catalyzed. In addition, enzymatic methods for the catalysis of the transesterification reaction are also known. An overview of the processes used to date for the industrial scale transesterification of oils to obtain fatty acid esters usable as fuel are given, for example, by the publications DE 199 25 871 A1, DE 602 09 028 T2 and DE 10 2004 044 660 A1, together with the references cited therein.

A problem common to all transesterification processes known to date is to conduct the purification of the product mixture from electrolyte-laden glycerol-containing phase in a very simple manner, such that especially the glycerol can be recovered only with remaining traces of water, electrolytes and by-products. The known processes are based, especially on the industrial scale, on the removal of the glycerol by means of distillation with the disadvantages described above.

It is therefore an object of the present invention to provide a simplified process for purifying an electrolyte-containing product from the mixture of a transesterification reaction in very high quality taking account of the requirement to incorporate the process especially into a continuous overall process on the industrial scale.

This object is achieved in accordance with the invention by a process for purifying the product mixture of a transesterification reaction with a polar, electrolyte-containing organic phase, in which the electrolytes are removed from the permeating polar organic phase by means of a nanofiltration step.

Transesterification reactions are, as already described above, an industrially important class of organic reactions in which an ester is converted to another ester by exchange of the acid groups or by exchange of the alcoholic groups. When the transesterification is effected by exchanging the alcoholic groups, reference is also made to an alcoholysis. In the alcoholysis, the alcohol to be exchanged is generally added in excess in order to obtain a high yield of desired ester. This is because the transesterification reaction is an equilibrium reaction which is generally triggered merely by mixing the reactants. However, the reaction proceeds so slowly that a catalyst is required for acceleration for commercial purposes. One example of an economically significant transesterification reaction is the preparation of fatty acid esters of short-chain alcohols, in which transesterification of natural fats or oils, for example rapeseed oil or soya oil, affords a product mixture of different fatty acid esters which is usable as a fuel.

Fats and oils of biological origin consist predominantly of glycerides (mono-, di- and triglyceride). In practice, the Bradshaw process is frequently employed for the transesterification of fats and oils with methanol, as described, for example, in U.S. Pat. Nos. 2,271,619 and 2,360,844. However, a wide variety of different modifications to the process are common There is a wide variation in the range of catalysts used in particular. In addition to the base-catalyzed processes (cf., for example, J. Am. Oil Chem. Soc. 61 (1984), 343 or Ullmann, Enzyklopadie der Technischen Chemie, 4th Edition, Volume 11, page 432), acid-catalyzed processes and enzymatic methods are also conceivable.

According to the invention, the term “electrolyte-containing product” is understood to mean the proportion of the product mixture from the transesterification reaction which, as well as the electrolytes, also comprises those compounds which have one or more free hydroxyl or acid group. For example, these are glycerol and short-chain alcohols, and also, to a lesser degree, free fatty acids (FFA) and water. The electrolytes refer here and hereinafter to all salt-type organic or inorganic compounds. Examples include the catalysts used with preference in the transesterification reaction, such as sodium hydroxide or potassium hydroxide, ammonium compounds or sulphuric acid compounds. However, those salts which get into the reaction mixture as a constituent of the reactants used shall also be encompassed by the term electrolytes according to the present invention.

In a preferred embodiment of the invention, the electrolyte-containing product comprises methanol, ethanol, isopropanol, water and/or glycerol.

According to the invention, the term “nanofiltration” is understood to mean a pressure-driven membrane separation process which retains particles in the nanometre range. Such particles are, for example, divalent ions. By definition, nanofiltration is, based on its separating performance, positioned between reverse osmosis, in which all dissolved substances are retained, and ultrafiltration, in which larger particles, for example between 2 nm and 0.1 μm, can be removed. Compared to reverse osmosis, correspondingly coarser filters and lower working pressures are used in nanofiltration. It is possible to use either non-porous or porous membranes. According to the invention, the nanofiltration is not used in the known sense of separating electrolytes and dissolved substances from an aqueous phase. Instead, in the present case, an electrolyte-containing organic phase is subjected to a nanofiltration.

The filtration temperature is not critical and can be varied within wide ranges. In a preferred embodiment of the invention, the nanofiltration is carried out at a temperature between 15° C. and 90° C., preferably between 20° C. and 60° C., more preferably between 30° C. and 40° C.

With the process according to the invention, it is possible for the first time to provide a process, which can also be used on the industrial scale, for purifying electrolyte-containing organic product mixtures from transesterification reactions, which, as well as a considerably simplified process regime owing to the now continuously conductible process, permits simplified integration into the overall process.

The inventive removal of the electrolytes from the permeating organic phase by means of a nanofiltration step advantageously achieves the effect that the mixture passing through the membrane, which consists preferably of glycerol, methanol and water, has been freed virtually completely of the electrolytes.

Owing to the fact that membrane separation processes work without a change in phase, significantly less energy is required for the separation process than in the case of comparable separation processes with a change in phase, for example distillative processes. A further advantage is that the purification by means of nanofiltration allows a considerably simplified apparatus construction. In addition, the purification can be carried out at room temperature or slightly elevated temperature, which, as well as the energy saving addressed, proceeds significantly more gently for the organic products, such that they can be obtained with a greater quality yield.

Preference is given to conducting the process according to the invention in such a way that a reaction mixture for transesterification, composed of reactant ester, catalyst and alcohol, is first stirred in a reaction vessel over a particular time. The reaction temperature, pressure and reaction time are not critical and are selected such that very substantial conversion is achieved with minimum residence time. The product mixture thus formed is sent to a separating stage, which separates the nonpolar product ester phase from the polar organic phase comprising the alcohol, glycerol and catalyst components. The polar organic phase removed, comprising the alcohol, glycerol and catalyst components, can be worked up in further steps, which, for example, first provide for a neutralization of the basic or acidic catalysts used. The neutralization step converts the bases or acids used as the catalyst to salts. The salt burden thus generated can subsequently be removed in accordance with the invention. In addition, the salt burden can optionally be reduced by simple removal of the already precipitated salt before the nanofiltration step.

Thus, in accordance with the invention, the organic phase obtained from the neutralization is subjected to a nanofiltration. According to the invention, filtration through a suitable non-porous or porous membrane at a filtration pressure between 5 bar and 70 bar, preferably between 10 bar and 60 bar, more preferably between 15 bar and 50 bar, affords an almost completely electrolyte-free mixture. The mixture may preferably consist of glycerol, short-chain alcohols such as methanol, ethanol and/or isopropanol, and small traces of water.

The term “almost completely electrolyte-free” is understood here and hereinafter to mean that an electrolyte content of below 0.01% by weight, based on divalent ions in the mixture, may be present. Preferably only an electrolyte content of below 0.005% by weight, based on divalent ions, is detectable in the permeated mixture.

The retained salt, which is present in concentrated form in a residual fraction of the mixture, can, for example, be added in the biodiesel process to the glycerol acidification, i.e. the removal stage of free fatty acids, salt and glycerol phase. This avoids additional use of assistants or the generation of additional waste streams.

The membrane used for the nanofiltration may consist of a porous or non-porous membrane known to those skilled in the art. The membrane used is preferably a polyamide-based membrane, for example a DOW Filmtech NF membrane, and may especially have a pore size of 0.005 μm to 0.1 μm, more preferably of 0.01 μm to 0.05 μm.

Moreover, the membrane should be selected such that, preferably, against the background of an industrial scale configuration, there is a high permeation flux and good stability of the membrane.

In a particular embodiment of the process, the membrane is a polymer membrane. The polymer membranes used are especially membranes based on polyamide, polysulphone, polyether sulphone, cellulose triacetate, cellulose acetate, thin film composites, silicones and combinations of these compounds.

In a preferred embodiment of the invention, polar organic solvents and/or water can be added before the filtration.

In this way, the viscosity of the mixture to be filtered can be adjusted to the optimal requirements of the process regime by simple addition of short-chain alcohols, for example methanol or ethanol.

The process according to the invention can be conducted either as a batchwise process or as a continuous process. It is more preferably conducted as a continuous process.

A process according to the present invention may especially preferably be used to obtain glycerol from the transesterification reaction of biological, i.e. vegetable and/or animal, fats or oils.

The invention is illustrated in detail by the examples which follow, without being restricted to them.

### EXAMPLES

### Example 1

A glycerol mixture comprising water, methanol, K₂SO₄ was passed through a crossflow system. The membrane used was the DK2540F1073 membrane from GE Osmonics. The membrane was used with a membrane area of 44 cm² and a volume flow of 450 l/h. Application of a transmembrane pressure of 59 bar allowed a permeate which has a mean conductivity of 0.31 mS to be achieved. The corresponding feed, which was circulated, had a mean conductivity of 2.7 mS.

The analytical testing of the samples of the two solutions gave the following measurement data:

TABLE 1

| | Feed | Permeate |
|---|---|---|
| Sulphate [% by wt.]* | 0.42 | 0.001 |
| Potassium [% by wt.]* | 0.43 | 0.037 |
| Water [% by wt.] | 42.3 | 46.4 |
| Methanol [% by wt.] | 12.5 | 15.1 |
| Glycerol [% by wt.] | 36.4 | 33.0 |
| Soaps [meq/kg] | 11.6 | 8.4 |
| FFA [meq/kg] | 3.5 | <1 |

As is clearly evident from Table 1, the electrolyte ions of the organic feed are effectively retained by the nanofiltration, such that the permeate can be obtained in almost electrolyte-free form even after a single filtration step.

### Example 2

A glycerol mixture comprising water, methanol and K₂SO₄ was passed through a crossflow system. The membrane used was an NF membrane from DOW Filmtech. The membrane was tested with a membrane area of 44 cm² and a volume flow of 410 l/h. The application of a transmembrane pressure of 20 bar allowed a permeate which has a mean conductivity of 0.49 mS to be achieved. The corresponding feed, which was circulated, had a mean conductivity of 2.8 mS. The analytical testing of samples of the two solutions gave the following measurement data:

TABLE 2

| | Feed | Permeate |
|---|---|---|
| Sulphate [% by wt.]* | 0.43 | 0.003 |
| Potassium [% by wt.]* | 0.44 | 0.060 |
| Water [% by wt.] | 42.9 | 44.2 |
| Methanol [% by wt.] | 13.0 | 13.7 |
| Glycerol [% by wt.] | 36.7 | 35.2 |
| Soaps [meq/kg] | 12.1 | 12.7 |
| FFA [meq/kg] | 3.3 | <1 |

Just like from Table 1, it is clearly evident from Table 2 that the electrolyte ions of the organic feed are effectively retained by the nanofiltration, such that the permeate can be obtained in almost electrolyte-free form even after a single filtration step.

1. A process for purifying the product mixture of a transesterification reaction with a polar, electrolyte-containing organic phase, wherein the electrolytes are removed from the permeating polar organic phase by means of a nanofiltration step.

2. A process according to claim 1, wherein the polar organic phase comprises glycerol, short-chain alcohols and water.

3. A process according to claim 2, wherein the polar organic phase comprises glycerol and methyl alcohol, ethyl alcohol and/or isopropyl alcohol.

4. A process according to claim 2, wherein the membrane used for the nanofiltration is a porous or a non-porous membrane.

5. A process according to claim 1, wherein the filtration pressure of the nanofiltration is between 5 bar and 70 bar.

6. A process according to any one of the preceding claims, wherein the nanofiltration is carried out at a temperature between 15° C. and 90° C.

7. A process according to claim 1, wherein polar organic solvents and/or water are added before the filtration.

8. A process according to claim 1, wherein the process is continuous it is conducted as a continuous process.

9. A process according to claim 2 for obtaining glycerol from the transesterification reaction of vegetable and/or animal fats or oils.

10. A process according to claim 5 wherein the filtration pressure of the nanofiltration is between 10 and 60 bar.

11. A process according to claim 10 wherein the filtration pressure of the nanofiltration is between 15 and 50 bar.

12. A process according to claim 6 wherein the nanofiltration is carried out at a temperature between 20° C. and 60° C.

13. A process according to claim 12 wherein the nanofiltration is carried out at a temperature between 30° C. and 40° C.
